SEQUENCE LISTING (8 sequences)

SEQ ID NO: 1 - DNA, 1470 bp, synthetic construct
  source 1..1470: mol_type = other DNA
atgagttataataaagaaaacaaaatgacatcagtatcaggcagacctatagctgataatcaaaattccctaactgctggcaaaagaggaccaatgcttttacaagatatttggtttttagaaaaactagctcattttgatagggaagtaattcctgaaagacgtatgcacgcaaaaggttcagctgcatttggaacgtttaccgttacacatgatattacccagtataccaaagctaaaatattttctgaaattggcaaacaaacagatttatttttaaggttttcaacagttgctggagaacgcggagcagctgatgcagaaagagatattcgtggttttgcagtgaaattctacactgaagaaggcaattgggacttagtaggaaacaatactcctgtattttttattcgtgatccgtataaatttccagatttaaacaaagcggtaaaaagagatcctggaacaaatcttagaagtgctaataataattgggattattggacattgttacccgaatctttacatcaaatcaccatgactatgagtgaacgtggtattcctaaaagttaccgtcacatgaatggttatggtagccatacatttagctttataaatgctcacaatgagcgtttttgggtgaaatttcattttaaaacagctcaaggaatagaatgtttaacggatcaagaatcagaaataataataggaaaagatagagaaagtcatcaaagagacttatacaacagcatcaaagaaggaaattatccaaaatggcacctgaaagtacaaataatgcctgaagaagatgctaaaacatatccttttaatccttttgatttaacaaaagtttggcctcatagtgattatcctcttattgatgttggagtattagaattaaataaaaatcctgaaaactattttgcagacgtagagcaatctgcctttaacccagcaaacatcgttcctggtataggcttttctcccgatagaatgttacaaggtagactcttttcttacggagatacacaacgctacagattaggagttaatcattatcaaatccctgtaaacgccccaagatgtccgtttcataatcatcataaagatggtgctatgcgtattgacggtaacggcggaggaacagtacattatgaacctaatagttatggaaaatggcaagaacaaaaagaatatagagaacctgctttagctctggaaggagctgctgatcattgggactttagagaagatgataatgattactacacacaaccaggtaacctatttagaatcatgactcctgaacaacaacaaacattgtttgaaaacacagcacgttctgtaggaggagctgaaagatttatacaagaacgtcatattcataattgttacaaagcagaccctaactatggcaaaggagttgccgcagctttaggcatcagcttaaccgatttagatttaatataa

SEQ ID NO: 2 - protein, 489 aa, synthetic construct
MSYNKENKMTSVSGRPIADNQNSLTAGKRGPMLLQDIWFLEKLAHFDREVIPERRMHAKGSAAFGTFTVTHDITQYTKAKIFSEIGKQTDLFLRFSTVAGERGAADAERDIRGFAVKFYTEEGNWDLVGNNTPVFFIRDPYKFPDLNKAVKRDPGTNLRSANNNWDYWTLLPESLHQITMTMSERGIPKSYRHMNGYGSHTFSFINAHNERFWVKFHFKTAQGIECLTDQESEIIIGKDRESHQRDLYNSIKEGNYPKWHLKVQIMPEEDAKTYPFNPFDLTKVWPHSDYPLIDVGVLELNKNPENYFADVEQSAFNPANIVPGIGFSPDRMLQGRLFSYGDTQRYRLGVNHYQIPVNAPRCPFHNHHKDGAMRIDGNGGGTVHYEPNSYGKWQEQKEYREPALALEGAADHWDFREDDNDYYTQPGNLFRIMTPEQQQTLFENTARSVGGAERFIQERHIHNCYKADPNYGKGVAAALGISLTDLDLI

SEQ ID NO: 3 - DNA, 1502 bp, synthetic construct
  source 1..1502: mol_type = other DNA
cgcgaacagattggaggtatgtcttataataaggaaaataagatgacatccgtgtcgggcagacctatagccgataaccagaatagtttgaccgccggaaagcgtggtcctatgttattacaagacatatggtttctggagaaacttgcacattttgatcgggaagtgatacctgaacgcagaatgcacgccaaaggatccgccgctttcgggacattcactgtcacccacgatataacccagtacacaaaggccaaaatattcagtgagattgggaaacagacagacctgtttctgcggttttcaacagtagcaggtgaaagaggtgcagctgacgcggagcgtgatatccgtgggtttgctgtaaagttttacaccgaggagggtaactgggatctggttggaaacaatactcctgtgttctttatacgcgacccatataaatttccggatttgaacaaagctgtcaagcgcgatccgggtaccaacttacgtagtgccaataataattgggactactggacccttctgcctgaatcgcttcaccaaattacaatgactatgtctgagcgcgggatccctaaatcctaccgccatatgaatggttacgggtcacacaccttttcgtttatcaatgcacataatgagcgcttctgggtaaagtttcattttaagacggcccaagggatcgaatgcttgaccgaccaagagagcgagataattatcggtaaagatagagagagccaccaacgcgatttatataatagcattaaggaaggaaattaccctaaatggcatttaaaagtccaaataatgcccgaagaggatgcaaagacctacccctttaacccgtttgatctgactaaggtctggccgcacagcgactatccgcttattgatgtcggggtcttggagctgaataaaaatcccgagaattattttgcggacgtagaacagtccgcattcaatcccgccaacattgtcccaggcattggcttctccccggatcggatgctgcaaggtagactgttttcctatggagacactcaacggtatcgcttaggtgtaaaccactatcagatacctgtgaatgccccacggtgcccgtttcataatcatcacaaggatggagcaatgcgtattgacggtaatgggggcggtaccgtccattatgaacctaatagctacggcaaatggcaggagcaaaaggaatatcgcgagcccgcgttggcccttgaaggtgctgcagatcattgggattttcgcgaagacgacaatgactactatacccaacctggaaatctttttagaataatgacacctgaacaacaacaaacgttgtttgaaaacacagctagatcagtcggtggggctgaacgtttcatacaggagcgtcatatacataactgttataaagccgaccccaattacggaaagggggtggctgcggcgttgggtatttctttaacagacttggacttgatataatagagcggccgcca

SEQ ID NO: 4 - protein, 495 aa, synthetic construct
REQIGGMSYNKENKMTSVSGRPIADNQNSLTAGKRGPMLLQDIWFLEKLAHFDREVIPERRMHAKGSAAFGTFTVTHDITQYTKAKIFSEIGKQTDLFLRFSTVAGERGAADAERDIRGFAVKFYTEEGNWDLVGNNTPVFFIRDPYKFPDLNKAVKRDPGTNLRSANNNWDYWTLLPESLHQITMTMSERGIPKSYRHMNGYGSHTFSFINAHNERFWVKFHFKTAQGIECLTDQESEIIIGKDRESHQRDLYNSIKEGNYPKWHLKVQIMPEEDAKTYPFNPFDLTKVWPHSDYPLIDVGVLELNKNPENYFADVEQSAFNPANIVPGIGFSPDRMLQGRLFSYGDTQRYRLGVNHYQIPVNAPRCPFHNHHKDGAMRIDGNGGGTVHYEPNSYGKWQEQKEYREPALALEGAADHWDFREDDNDYYTQPGNLFRIMTPEQQQTLFENTARSVGGAERFIQERHIHNCYKADPNYGKGVAAALGISLTDLDLI

SEQ ID NO: 5 - DNA, 480 bp, synthetic construct
  source 1..480: mol_type = other DNA
atgaaaataaataatttaggattaccagtagaagaaacaaaagaattagttgtgcaactaaataagttgttgtctaactttcaaatttattatcaaaatttgagaggtttacactggaatatacgtggaaagcgattttttgacttgcatctaaaatttgaagaactctataatgatgcacaacttaaaatagatatgatagcagagcgtattttaactttagatggagttccttatcatacttttgaagactatttaaaaaatagtgaacttacagtaggtaaaaaaatttcaaatgacgaaaaggcaattagacttattgtagaatctattactactttgttagtaattgaacgtaaaatcttaaaagactcagatgttatagatgatgaaggtaccagttcaatgatgagcgattttataaaagaacaagagaagactatttggatgatgagtgcttggttagaagaaacgatggat

SEQ ID NO: 6 - protein, 160 aa, synthetic construct
MKINNLGLPVEETKELVVQLNKLLSNFQIYYQNLRGLHWNIRGKRFFDLHLKFEELYNDAQLKIDMIAERILTLDGVPYHTFEDYLKNSELTVGKKISNDEKAIRLIVESITTLLVIERKILKDSDVIDDEGTSSMMSDFIKEQEKTIWMMSAWLEETMD

SEQ ID NO: 7 - DNA, 516 bp, synthetic construct
  source 1..516: mol_type = other DNA
cgcgaacagattggaggtatgaaaatcaataacctgggtctgccagtggaagagacaaaagaacttgtggttcaactgaacaaacttctgtccaacttccaaatctactaccagaatttgcgcggattgcattggaacatacgcgggaagcgtttcttcgaccttcatttgaagtttgaagaactgtataatgacgcacagcttaaaatcgacatgattgccgagcggatactgacccttgatggcgtcccgtaccatacttttgaggattatttgaagaactcagaactgacagtggggaagaaaatatctaacgatgaaaaggcaattcggcttatcgtagaaagcatcacaacacttcttgtaatcgaacgcaagatattaaaggattctgatgtaattgatgatgaaggcacaagtagtatgatgtcggatttcattaaagagcaggagaagaccatatggatgatgagtgcttggttagaagagactatggattaatagagcggccgccac

SEQ ID NO: 8 - protein, 166 aa, synthetic construct
REQIGGMKINNLGLPVEETKELVVQLNKLLSNFQIYYQNLRGLHWNIRGKRFFDLHLKFEELYNDAQLKIDMIAERILTLDGVPYHTFEDYLKNSELTVGKKISNDEKAIRLIVESITTLLVIERKILKDSDVIDDEGTSSMMSDFIKEQEKTIWMMSAWLEETMD

(19) United States
(12) Patent Application Publication
(10) Pub. No.: US 2025/0340601 A1
(43) Pub. Date: Nov. 6, 2025

(54) RECOMBINANT FLAVOBACTERIUM COVAE PROTEIN VACCINES

(71) Applicant: The United States of America, as Represented by the Secretary of Agriculture, Washington, DC (US)

(72) Inventor: MILES D LANGE, OPELIKA, AL (US)

(21) Appl. No.: 19/077,425

(22) Filed: Mar. 12, 2025

Related U.S. Application Data
(60) Provisional application No. 63/641,586, filed on May 2, 2024.

(51) Int. Cl. C07K 14/195; A61K 39/00

(52) U.S. Cl. CPC C07K 14/195; A61K 39/00; A61K 2039/542; A61K 2039/552; A61K 2039/70

(57) ABSTRACT

The instant disclosure provides vaccines for fish to Flavobacterium covae by utilizing one or more recombinant F. covae catalase and DPS proteins. Isolated and expressed in recombinant host cells, these proteins are formulated for delivery to fish (e.g., catfish) as an immunostimulatory composition resulting in increased resistance to F. covae infection.

### CROSS-REFERENCE

The present application claims priority to U.S. Provisional Patent Application Ser. No. 63/641,586 filed May 2, 2024, the contents of which are expressly incorporated herein by reference.

### SEQUENCE LISTING

The instant application contains a Sequence Listing XML required by 37 C.F.R. § 1.831(a) which has been submitted in XML file format via the USPTO patent electronic filing system and is hereby incorporated by reference in its entirety. The XML file was created on Apr. 23, 2024, is named Sequence_Listing-000224.xml, and has 12.7 KB.

### BACKGROUND OF THE INVENTION

### Field of Invention

The instant disclosure provides vaccines for fish to Flavobacterium covae by utilizing one or more recombinant F. covae catalase and DPS proteins. Isolated and expressed in recombinant host cells, these proteins are formulated for delivery to fish (e.g., catfish) as an immunostimulatory composition resulting in increased resistance to F. covae infection.

### Background

The catfish industry is comprised of channel and hybrid catfish and is the largest sector of U.S. aquaculture ($450 million in food-size catfish in 2021). Columnaris disease is one of the leading causes of mortality in the production of freshwater farmed finfish species and produces approximately $2 million dollars in annual losses and $15 million (2015-2021) in the U.S. (Declercq et al, Vet. Res., (2013), 44:1-17; Abdelrahman et al, Aquaculture, (2023), 566:739206). This is due to the ubiquitous bacterium Flavobacterium columnare that is commonly found in the aquatic environment. Recently, LaFrentz et al. reclassified F. columnare into four distinct species: F. columnare, F. covae, F. davisii, and F. oreochromis. F. covae is shown to have specific host-associations with channel catfish (LaFrentz et al, Front. Microbiol., (2018), 9:1-13; LaFrentz et al, Syst. Appl. Microbiol., (2021), 45:126293). As food fish production continues to increase, the frequency of columnaris disease will only continue to rise within the aquaculture industry. Add to this an increase in the regulation of treatments and resistance to available antibiotics means that alternative methods of disease protection will be required.

Early research in different fish species examined the used of F. columnare bacterins with some success. In the early 2000's, a modified live vaccine for F. columnare was developed, licensed, and used by the catfish industry using an F. columnare isolate (Shoemaker et al, Fish Shellfish Immunol., (2011), 30:304-8). Although this vaccine was proven effective in the laboratory, the efficacy under production conditions was variable and use of the vaccine declined (Bebak & Wagner, J. Aquatic Animal Health, (2012), 24:30-6). The research on this vaccine perpetuated subsequent research on other modified live vaccines with similar results in the laboratory (Mohammed et al, Vaccine, (2013), 31:5276-80).

### SUMMARY OF THE INVENTION

The present disclosure provides a composition comprising an isolated protein having an amino acid sequence at least 75% identical to SEQ ID NO: 2, SEQ ID NO: 6, or a combination of these two proteins, and an adjuvant. In some embodiments, the protein is at least 75% identical to SEQ ID NO:2. In some additional embodiments, the protein is at least 75% identical to SEQ ID NO:6.

The present disclosure further provides a composition comprising an isolated protein having an amino acid sequence at least 75% identical to SEQ ID NO: 2 and an isolated protein having an amino acid sequence at least 75% identical to SEQ ID NO: 6. In some embodiments, this composition comprises an adjuvant.

The instant disclosure further provides, a vaccine composition comprising an effective amount of a protein having an amino acid sequence at least 75% identical to SEQ ID NO: 2, SEQ ID NO: 6, or a combination thereof, and an adjuvant. In some embodiments, the protein is at least 75% identical to SEQ ID NO:2. In additional embodiments, the protein is at least 75% identical to SEQ ID NO:6. In another embodiment, the vaccine comprises an isolated protein having an amino acid sequence at least 75% identical to SEQ ID NO: 2 and an isolated protein having an amino acid sequence at least 75% identical to SEQ ID NO: 6.

Further disclosed herein is, an expression vector comprising a promoter and a heterologous polynucleotide, wherein the heterologous polynucleotide encodes a protein having an amino acid sequence at least 75% identical to SEQ ID NO: 2 or SEQ ID NO: 6, and wherein the promoter is operatively linked to the heterologous polynucleotide.

The instant disclosure also provides a method of eliciting an immune response against Flavobacterium covae in a subject, by administering the composition comprising a protein at least 75% identical to SEQ ID NO: 2 or SEQ ID NO: 6, thereby eliciting an immune response to F. covae in the subject. In some embodiments, the protein comprises SEQ ID NO: 2 or SEQ ID NO: 6. In a particular embodiment, the administered composition comprises two proteins, wherein the first protein is at least 75% identical to SEQ ID NO: 2 and the second protein is at least 75% identical to SEQ ID NO: 6. In some embodiments, the composition further comprises a pharmaceutically acceptable carrier, an adjuvant, or both a pharmaceutically acceptable carrier and an adjuvant. In some embodiments, the subject is a fish, such as a catfish. In some embodiments, the administration of the protein is via the oral route.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

The novel features of the invention are set forth with particularity in the claims. Features and advantages of the present invention are referred to in the following detailed description, and the accompanying drawings of which:

FIG. 1 provides photographic data of recombinant expression of F. covae proteins. Coomassie stain of Ni-NTA purified catalase (lane 1) and DPS (lane 2) after 24 h of IPTG stimulation. The arrows mark the 56 and 22 kDa bands representing the approximate size of the eluted catalase and DPS proteins, respectively. (M) is the pre-stained Precision Plus marker (kDa) used to estimate molecular mass.

FIG. 2A and FIG. 2B provide representation of adaptive immune response to recombinant F. covae catalase. FIG. 2A depicts catalase specific IgM antibodies in the serum as determined by ELISA. The mean±SE for each group is shown as a horizontal line. The dashed line represents background absorbance with the secondary HRP Ab only. Differences were considered significant, * (P<0.05). FIG. 2B is a photograph of an immunoblot of recombinant catalase; lanes 2 and 3 are two individual fish, lane 4 is the secondary HRP antibody (Ab) only and lane 1 is the pre-stained Western C marker (kDa) used to estimate molecular mass. Fish C1 (grey) and C2 (black) are represented by squares in panels A and B.

FIG. 3A and FIG. 3B provide representation of adaptive immune response to recombinant F. covae DPS. (A) DPS-specific IgM antibodies in the serum as determined by ELISA. The mean±SE for each group is shown as a horizontal line. The dashed line represents background absorbance with the secondary HRP Ab only. Differences were considered significant, * (P<0.05). (B) Immunoblot of recombinant catalase; lanes 2 and 3 are two individual fish, lane 4 is the secondary HRP Ab only and lane 1 is the pre-stained Western C marker (kDa) used to estimate molecular mass. Fish D2 (grey) and D3 (black) are represented by triangles in panels A and B.

FIG. 4A and FIG. 4B provide representation of adaptive immune response to recombinant F. covae DPS. FIG. 4A: Catalase-specific IgM antibodies in the skin as determined by ELISA. The mean±SE for each group is shown as a horizontal line. FIG. 4B: DPS-specific IgM antibodies in the skin as determined by ELISA. For both panels the mean absorbance±SE for each group is shown as a horizontal line The dashed line represents background absorbance with the secondary HRP Ab only.

FIG. 5 provides depiction of a Kaplan-Meier survival curve of channel catfish challenged with isolate LV-359-01 9-weeks post immunization with recombinant F. covae proteins. The different vaccine and control groups are labeled as indicated in the legend. Data represent cumulative mortality across replicate aquaria per group containing 100 fish (n=25/aquaria). Asterisk(s) denote a significant (*) or very significant (**) difference in survival when compared to the adjuvant only control; P<0.05.

FIG. 6 provides a depiction of relative expression of innate immune genes in channel catfish skin after immunization with F. covae recombinant protein vaccines. Data are presented as mean±SE, and the 18S reference gene was used to normalize with the target gene (n=4). The solid line represents the adjuvant only control gene expression set to 1-fold. Different letters indicate significant differences between the vaccine groups when compared to the adjuvant control (P<0.01).

FIG. 7 provides depiction of relative expression of B-cell mediated genes in channel catfish skin after immunization with F. covae recombinant protein vaccines. Data are presented as mean±SE, and the 18S reference gene was used to normalize with the target gene (n=4). The solid line represents the adjuvant only control gene expression set to 1-fold. Different letters indicate significant differences between the vaccine groups when compared to the adjuvant control (P<0.01).

FIG. 8 provides depiction of relative expression of T-cell mediated genes in channel catfish skin after immunization with F. covae recombinant protein vaccines. Data are presented as mean±SE, and the 18S reference gene was used to normalize with the target gene (n=4). The solid line represents the adjuvant only control gene expression set to 1-fold. Different letters indicate significant differences between the vaccine groups when compared to the adjuvant control (P<0.01).

FIG. 9 provides depiction of Kaplan-Meier survival curve of channel catfish challenged with F. covae ALG-00-530 isolate six weeks post immunization with different oral doses of the recombinant F. covae catalase (SEQ ID NO:2) protein. The different control and vaccinated groups are labeled as indicated. Data represent cumulative mortality across four replicate aquaria per group containing 80 fish (n=20/aquaria). Asterisks denote a very significant (**) difference in survival when compared to the adjuvant only control; P<0.05.

FIG. 10 provides depiction of Kaplan-Meier survival curve of channel catfish challenged with F. covae ALG-00-530 isolate six weeks post oral immunization with different doses of the recombinant F. covae DPS (SEQ ID NO:6) protein. The different vaccine and control groups are labeled as indicated in the legend. Data represent cumulative mortality across four replicate aquaria per group containing 80 fish (n=20/aquaria). Asterisk denotes a significant (*) difference in survival when compared to the adjuvant only control; P<0.05.

### DETAILED DESCRIPTION OF THE INVENTION

In this work, we sought to characterize the catfish adaptive immune response to F. covae and have found that there is a wide array of low and high adaptive immune responders to columnaris disease. This response includes the production of anti-F. covae antibodies to different bacterial cellular and extracellular proteins (Lange et al, Fish Shellfish Immunol., (2016), 51:170-9). This invention disclosure covers the development and use of F. covae catalase (e.g., SEQ ID NO: 2) and DNA starvation/stationary phase protein (DPS) (e.g., SEQ ID NO: 6) as recombinant protein vaccines for the protection against columnaris disease.

Preferred embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby.

Technical and scientific terms used herein have the meanings commonly understood by one of ordinary skill in the art to which the instant invention pertains, unless otherwise defined. Reference is made herein to various materials and methodologies known to those of skill in the art. Standard reference works setting forth the general principles of recombinant DNA technology include Sambrook et al., “Molecular Cloning: A Laboratory Manual”, 2d ed., Cold Spring Harbor Laboratory Press, Plainview, N.Y., 1989; Kaufman et al., eds., “Handbook of Molecular and Cellular Methods in Biology and Medicine”, CRC Press, Boca Raton, 1995; and McPherson, ed., “Directed Mutagenesis: A Practical Approach”, IRL Press, Oxford, 1991. Standard reference literature teaching general methodologies and principles of fungal genetics useful for selected aspects of the invention include: Sherman et al. “Laboratory Course Manual Methods in Yeast Genetics”, Cold Spring Harbor Laboratory, Cold Spring Harbor, N.Y., 1986 and Guthrie et al., “Guide to Yeast Genetics and Molecular Biology”, Academic, New York, 1991.

Any suitable materials and/or methods known to those of skill can be utilized in carrying out the instant invention. Materials and/or methods for practicing the instant invention are described. Materials, reagents and the like to which reference is made in the following description and examples are obtainable from commercial sources, unless otherwise noted. This invention teaches methods and describes tools for.

As used in the specification and claims, use of the singular “a”, “an”, and “the” include plural references unless the context clearly dictates otherwise.

The terms isolated, purified, or biologically pure as used herein, refer to material that is substantially or essentially free from components that normally accompany the referenced material in its native state.

The term “about” is defined as plus or minus ten percent of a recited value. For example, about 1.0 g means 0.9 g to 1.1 g and all values within that range, whether specifically stated or not.

The term “adjuvant” means a substance or vehicle that non-specifically enhances the immune response to an antigen. Adjuvants can include a suspension of minerals (alum, aluminum hydroxide, or phosphate) on which antigen is adsorbed; or water-in-oil emulsion in which antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (see, e.g., U.S. Pat. Nos. 6,194,388; 6,207,646; 6,214,806; 6,218,371; 6,239,116; 6,339,068; 6,406,705; and 6,429,199). Adjuvants also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL. Any veterinarily accepted adjuvants can be utilized, such as Montanide ISA and IMS Adjuvants, Ribi's Adjuvants (Ribi ImmunoChem Research, Inc., Hamilton, MT), Hunter's TiterMax (CytRx Corp., Norcross, GA), aluminium salt adjuvants, nitrocellulose-adsorbed proteins, encapsulated antigens, nanoparticle containing adjuvants.

The term “administer” /“administration” means any method of providing a subject with a substance, such as a therapeutic agent by any effective route. Useful effective routes are readily determined by the skilled artisan and include techniques now known and those developed in the future.

The term “antibody” refers to an immunoglobulin molecule produced by B lymphoid cells with a specific amino acid sequence. Antibodies are evoked in humans or other animals by a specific antigen (immunogen). Antibodies are characterized by reacting specifically with the antigen in some demonstrable way, thus, antibody and antigen are at least partially defined in terms of the other.

The term “antigen” refers to a substance that is able to induce a humoral antibody and/or cell-mediated immune response rather than immunological tolerance. The term signifies the ability to stimulate an immune response as well as react with the products of it, e.g., an antibody.

“Carrier” as used herein refers to any method of dispersal, dispensation, application, timed-release, encapsulation, microencapsulation, or the like to apply the antigen compositions as further described herein. In embodiments, such “carriers” may include a variety of microencapsulation, controlled release, and other dispersion technologies available to those of ordinary skill in the art.

The term “catalase” refers to the F. covae-derived protein defined herein as SEQ ID NO: 2 or SEQ ID NO:4 and encoded by the DNA of SEQ ID NO: 1 or SEQ ID NO:3 (or any version of SEQ ID NO: with base substitutions that result in a protein with a sequence identical to SEQ ID NO:1 or SEQ ID NO:3). This term, in context, can also refer to antigenic portions of the reference protein.

The term “DPS” refers to the F. covae-derived protein defined herein as SEQ ID NO:6 or SEQ ID NO:8 and encoded by the DNA of SEQ ID NO:5 or SEQ ID NO:7 (or any version of SEQ ID NO:2 or SEQ ID NO: 7 with base substitutions that result in a protein with a sequence identical to SEQ ID NO:6 or SEQ ID NO:8). This term, in context, can also refer to antigenic portions of the reference protein.

The term “control”, and grammatical variants thereof, is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

The term “effective amount” of a composition provided herein refers to the amount of the composition capable of performing the specified function for which an effective amount is expressed. The exact amount required can vary from composition to composition and from function to function, depending on recognized variables such as the compositions and processes involved. An effective amount can be delivered in one or more applications. Thus, it is not possible to specify an exact amount, however, an appropriate “effective amount” can be determined by the skilled artisan via routine experimentation.

The term “immune response” refers to a response of a cell of the immune system, such as a B-cell, T-cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate immune response or inflammation. As used herein, a protective immune response refers to an immune response that protects a subject from infection (prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, antibody production and the like.

A first nucleic acid sequence is “operably linked” with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

The terms “polypeptide”, “peptide”, and “protein” refer to polymers in which the monomers are amino acid residues which are joined together through amide bonds. When the amino acids are alpha-amino acids, either the L-optical isomer or the D-optical isomer can be used. The terms are used interchangeably herein. These terms apply to amino acid polymers in which one or more amino acid residues are an artificial chemical mimetic of a corresponding naturally occurring amino acid, as well as to naturally occurring amino acid polymers and non-naturally occurring amino acid polymers.

As used herein, the term “promoter” refers to a polynucleotide that in its native state is located upstream or 5′ to a translational start codon of an open reading frame (or protein-coding region) and that is involved in recognition and binding of RNA polymerase and other proteins (trans-acting transcription factors) to initiate transcription. The term can include promoters produced through the manipulation of known promoters to produce artificial, chimeric, or hybrid promoters. Such promoters can also combine cis-elements from one or more promoters, for example, by adding a heterologous regulatory element to an active promoter with its own partial or complete regulatory elements. The term “cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch, J. Mol. Biol., (1970) 48:3, 443-53). A computer-assisted sequence alignment can be conveniently performed using a standard software program such as GAP which is part of the Wisconsin Package Version 10.1 (Genetics Computer Group, Madison, Wisconsin, USA) using the default scoring matrix with a gap creation penalty of 50 and a gap extension penalty of 3.

The phrase “high percent identical” or “high percent identity”, and grammatical variations thereof in the context of two polynucleotides or polypeptides, refers to two or more sequences or sub-sequences that have at least about 80%, identity, at least about 81%, 82%, 83%, 84%, 85%, 86%, 87%, 88%, 89%, 90%, 91%, 92%, 93%, 94%, 95%, 96%, 97%, 98%, 99%, or 100% nucleotide or amino acid identity, when compared and aligned for maximum correspondence, as measured using a sequence comparison algorithm or by visual inspection. In an exemplary embodiment, a high percent identity exists over a region of the sequences that is at least about 16 nucleotides or amino acids in length. In another exemplary embodiment, a high percent identity exists over a region of the sequences that is at least about 50 nucleotides or amino acids in length. In still another exemplary embodiment, a high percent identity exists over a region of the sequences that is at least about 100 nucleotides or amino acids or more in length. In one exemplary embodiment, the sequences are high percent identical over the entire length of the polynucleotide or polypeptide sequences.

The term “vaccine” refers to a preparation of immunogenic material capable of stimulating an immune response, administered for the prevention, amelioration, or treatment of disease, such as an infectious disease. The immunogenic material can include, for example, attenuated or killed microorganisms (such as attenuated viruses), or antigenic proteins, peptides or DNA derived from an infectious microorganism. Vaccines can elicit both prophylactic (preventative) and therapeutic responses. Methods of administration vary according to the vaccine, but can include inoculation, ingestion, inhalation or other forms of administration. Inoculations can be delivered by any of a number of routes, including parenteral, such as intravenous, subcutaneous or intramuscular. Vaccines can be administered with an adjuvant to boost the immune response.

A “vector” is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An “expression vector” is a vector that contains the necessary regulatory sequences to allow transcription and translation of inserted gene or genes.

### Molecular Biological Methods

An isolated nucleic acid is a nucleic acid the structure of which is not identical to that of any naturally occurring nucleic acid. The term therefore covers, for example, (a) a DNA which has the sequence of part of a naturally occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transformed or transfected cells, and (iii) cell clones, e.g., as these occur in a DNA library such as a cDNA or genomic DNA library.

The term recombinant nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

In practicing some embodiments of the invention disclosed herein, it can be useful to modify the DNA of a recombinant strain of a host cell producing the immunogenic protein of the immunogenic compositions (e.g., the proteins of SEQ ID NO: 2 and/or SEQ ID NO: 6). In some embodiments, such a host cell is E. coli. Such modification can involve deletion of all or a portion of a target gene, including but not limited to the open reading frame of a target locus, transcriptional regulators such as promoters of a target locus, and any other regulatory nucleic acid sequences positioned 5′ or 3′ from the open reading frame. Such deletional mutations can be achieved using any technique known to those of skill in the art. Mutational, insertional, and deletional variants of the disclosed nucleotide sequences and genes (with resulting effects on the proteins expressed from these sequences) can be readily prepared by methods which are well known to those skilled in the art. It is well within the skill of a person trained in this art to make mutational, insertional, and deletional mutations.

Where a recombinant nucleic acid is intended for expression, cloning, or replication of a particular sequence, DNA constructs prepared for introduction into a prokaryotic or eukaryotic host will typically comprise a replication system (i.e. vector) recognized by the host, including the intended DNA fragment encoding a desired polypeptide, and can also include transcription and translational initiation regulatory sequences operably linked to the polypeptide-encoding segment. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See www.ncbi.nih.gov.

Any suitable bacterial, protist, animal or fungal host capable of expressing the described proteins can be utilized. Even more preferably, non-pathogenic and non-toxigenic strains of such host cells are utilized in practicing embodiments of the disclosed inventions. Examples of workable combinations of cell lines and expression vectors are described in Sambrook et al. (1989); Ausubel et al. (Eds.) (1995) Current Protocols in Molecular Biology, Greene Publishing and Wiley Interscience, New York; and Metzger et al. (1988) Nature, 334:31-36. Recombinant host cells, in the present context, are those which have been genetically modified to contain an isolated nucleic molecule, or produce a recombinant protein, of the instant invention. The nucleic acid(s) encoding the protein(s) of the present invention can be introduced by any means known to the art which is appropriate for the particular type of cell, including without limitation, transformation, lipofection, electroporation or any other methodology known by those skilled in the art.

### Vaccines (Immunogenic Compositions)

Administration of the vaccines (immunogenic compositions) result in increased immunity to a disease; the immunogenic compositions stimulate antibody production, cellular immunity, or both against the pathogen causing the disease. Immunity is defined herein as the induction of a significantly higher level of protection in a population of recipients, such as fish, against mortality and clinical symptoms after receipt of an immunogenic composition compared to an untreated group. In particular, the immunogenic composition(s) according to the invention can: (a) protect a large proportion of treated animals against the occurrence of clinical symptoms of the disease and mortality, or; (b) result in a significant decrease in clinical symptoms of the disease and mortality.

The immunogenic composition(s) of the invention herein, regardless of other components included, comprise a recombinant catalase protein from F. covae (e.g., SEQ ID NO: 2), a recombinant DPS protein from F. covae (e.g., SEQ ID NO: 6). Recombinant vaccine proteins of the present invention can comprise the entirety of SEQ ID NO: 2 (or antigenic portions thereof), SEQ ID NO: 6 (or antigenic portions thereof), and combinations thereof. Proteins of the present invention can also include those with 90%, 91%, 92%, 93%, 94%, 95%, 96%, 97%, 98%, 99%, or 100% homology to the protein of SEQ ID NO: 2 and the protein of SEQ ID NO: 6.

The immunogenically effective amounts of vaccines disclosed herein can vary based upon multiple parameters. In general, however, effective amounts per dosage unit can be about 10-200 μg recombinant protein, about 20-150 μg recombinant protein, or about 50-100 μg recombinant protein. An individual dose can contain 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 235, 240, 245, 250 or more μg of recombinant protein per dose. These amounts can also include antigenic portions of the full-length protein. In embodiments where a vaccine of the instant disclosure comprises more than one recombinant protein, the dosage units provided can refer to total protein content or the dosage units of the individual proteins within the composition.

One, two, or more dosage units can be utilized in practicing the methodologies of the present invention. If two dosage units are selected, then a booster dose can be applied as determined by the skilled artisan. A dosage unit can readily be modified to fit a desired volume or mass by one of skill in the art. Regardless of the dosage unit parameters, vaccines disclosed herein can be administered in an amount effective to produce an immune response to the presented antigen(s) (e.g., catalase or DPS protein).

Dosage levels of active ingredients (e.g., catalase and/or DPS protein) in vaccines disclosed herein, can be varied by one of skill in the art to achieve a desired result in a subject or per application. As such, a selected dosage level can depend upon a variety of factors including, but not limited to, formulation, combination with other treatments, severity of a pre-existing condition, and the presence or absence of adjuvants. In preferred embodiments, a minimal dose of an immunogenic composition is administered. As used herein, the term “minimal dose” or “minimal effective dose” refers to a dose that demonstrates the absence of, or minimal presence of, toxicity to the recipient, but still results in producing a desired result (e.g., protective immunity). Minimal effective doses, or minimum immunizing doses, of the recombinant immunogenic compositions provided herein can include about 10-200 μg recombinant protein, about 20-150 μg recombinant protein, or about 50-100 μg recombinant protein. The minimal effective doses can also be any dose within the range of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 200, 205, 210, 215, 220, 225, 230, 235, 240, 245, 250 or more μg of recombinant protein per dose. These amounts can also include antigenic portions of the full-length protein. Determination of a minimal dose is well within the capabilities of one skilled in the art. In embodiments where a vaccine of the instant disclosure comprises more than one recombinant protein, the dosage units provided can refer to total protein content or the dosage units of the individual proteins within the composition.

### Routes of Administration

The skilled artisan is able to administer vaccine preparations of the instant disclosure by any means known in the art and developed in the future. Some exemplary, but non-limiting, examples are provide here. Vaccination of large fish stocks with vaccines disclosed herein can be achieved by oral administration through the feed, for example, the vaccine can be incorporated within a feed product with, or without, protective coatings Vaccination can also be performed by injection, such as by hand or with the aid of machine injectors. In embodiments where large numbers of fish are to be vaccinated, vaccines of the instant disclosure can be dissolved, immersed, and/or dispersed into a body of water (e.g., a farm pond). Additionally, vaccinating large stocks of fish can be accomplished by spraying the fish temporarily removed from the water, with a composition comprising the vaccines of the instant invention.

The embodiments illustratively disclosed herein suitably may be practiced in the absence of any element [e.g., method (or process) steps or composition components)] which is not specifically disclosed herein. Thus, the specification includes disclosure by silence. Written support for a negative limitation may also be found through the absence of the excluded element in the specification, known as disclosure by silence.

Having generally described this invention, the same will be better understood by reference to certain specific examples, which are included herein to further illustrate the invention and are not intended to limit the scope of the invention as defined by the claims.

### EXAMPLES

### Example 1

Construction, Expression and Evaluation of F. covae Protein Vaccines.

To construct the expression vectors, we first retrieved the coding sequences from Flavobacterium covae strain 94-081; catalase (AWN65_03970) and DNA starvation/stationary phase protein (DPS) (AWN65_06620). The nucleotide sequences were submitted for codon optimization, incorporation of restriction sites, NcoI and XhoI, flanking the coding region, synthesis and cloning into pET-28a(+) expression vector with a C-terminal His-tag (Genscript, Piscataway, NJ). The codon optimized catalase coding sequence (SEQ ID NO: 3) shared 77% nucleotide identity to the F. covae coding sequence (SEQ ID NO: 1) and codon optimized DPS coding sequence (SEQ ID NO: 7) which shared 76% nucleotide identity to the F. covae coding sequence (SEQ ID NO: 5) from the F. covae strain 94-081 genome. Each sequence was verified by sanger sequencing, and plasmids were cloned into Escherichia coli strain BL21 (DE3) (Invitrogen, Carlsbad, CA) on Luria-Bertani (LB) agar supplemented with kanamycin (50μg/mL) and grown at 37° C. For expression, the E. coli expressing recombinant F. covae proteins were retrieved from frozen glycerol stocks stored at −80° C. and streaked onto LB agar plate and grown overnight. A single colony was then cultured overnight in 100 mL of LB broth supplemented with kanamycin (50 μg/mL). A flask containing 1 L of fresh LB broth with kanamycin was inoculated with the entire overnight culture and incubated for 2-4 h under the same culture conditions. Recombinant protein expression was induced by adding isopropyl thiogalactoside (IPTG) at a 1 mM final concentration when the absorbance (OD550) was >0.4; and then cultured for up to 24 h. F. covae recombinant proteins were purified under native conditions (Ni-NTA handbook, Qiagen) using a HisPur Ni-NTA agarose resin (ThermoFisher, Waltham, MA). Protein concentration was estimated using the BCA assay kit (ThermoFisher, Waltham, MA) with BSA as the standard. Absorbance was read at a wavelength of 562 nm with a Cytation 1 reader (Agilent, Santa Clara, CA) operating under Gen5 software. Aliquots were dispensed and kept at-20° C. until needed. SDS gel electrophoresis was conducted to analyze expressed protein using 10% TGX stain-free gels and buffers of the mini-protean system (Biorad, Hercules, CA). We loaded 5 μg of recombinant proteins onto SDS gels with the Precision Plus gel marker (Biorad, Hercules, CA), stained using Simple Blue Safe (ThermoFisher, Waltham, MA) and visualized using a ChemiDoc XRS+ gel system operating under Image Lab 3.0 software.

Delta select channel catfish fingerlings were reared at the Aquatic Animal Health Research Unit, Auburn, AL, USA. Channel catfish (5 g) were anesthetized by immersion into water contained buffered MS-222at 100-200 ppm prior to being immunized. The fish were injected intraperitoneally with the appropriate dose of 200 μg/mL F. covae recombinant protein (catalase or DPS) in 100 μL of (70:30) adjuvant (ISA 763AVG, SEPPIC, Fairfield, NJ) to antigenic medium (1× PBS and recombinant F. covae protein) using a 5/8 to1-inch (21-26 gauge) syringe just posterior and under the pelvic fin. Control fish received 100 μL of (70:30) adjuvant with empty antigenic medium (1× PBS). The fish were sampled 30 days post immunization.

Delta select channel catfish (5.5 g) were bath immunized with F. covae recombinant protein(s) and adjuvant (IMS1312 VG PR, SEPPIC, Fairfield, NJ) prior to being stocked into 300 L tanks that received a mix of filtered well/municipal water and aeration from submerged air stones. The vaccine groups were immunized in 50 L water with 1 μg/mL of recombinant protein(s) with adjuvant (catalase, DPS or catalase and DPS). Adjuvant only control fish were sham vaccinated in 50 L water with an adjuvant/1× PBS mixture. All fish were kept on flow through for 1 week post immunization and then all the tanks were switched over and maintained using a recirculating aquaculture system (RAS) supplied with mixed (well and dechlorinated municipal water) for the remainder of the study.

All experimental fish protocols used in this study were approved by the USDA-ARS Aquatic Animal Health Research Unit or the Auburn University Institutional Animal Care and Use Committees. Channel catfish from the adjuvant-only control, and the vaccinated groups (catalase, DPS, catalase/DPS) were sampled every two weeks for 13 weeks. Fish were euthanized and blood was collected using a 21-gauge needle from the caudal vein and allowed to clot overnight at 4° C. Blood samples were centrifuged at 10000×g for 5 min using a Legend Micro 21R centrifuge (Sorvall); the serum (50-300 μL) was removed and stored at −80° C. until needed. After blood collection we proceeded with the preparation of excised skin for tissue culture as described (Lange et al, (2016), supra). Briefly we wiped down the surface of the skin on both sides three times with a 70% ethanol solution. Then using sterile instruments, we dissected two 1.5 mm² skin pieces along the left lateral line and one 1.5 mm² skin piece along the right lateral line and placed the two-piece samples into 300 μL of complete RPMI 1640 medium with 10% DI water for catfish cell tonicity (10% FBS, penicillin/streptomycin, amphotericin, gentamicin) in a 48-well plate at 28° C. for 24 h. The next day the skin explant tissue culture medium was removed and stored at −80° C. until needed. A second dissection removed one 1.5 mm² skin piece along the right lateral line which was placed into RNAlater and stored at −80° C. until needed.

We used an indirect ELISA to measure the skin-based IgM antibodies, Immulon 2HB 96-well plates (ThermoFisher, Waltham, MA) were coated with 100 μL of 10 μg/mL of recombinant F. covae catalase or DPS in a sodium bicarbonate buffer. Plates were then rinsed three times with 1× PBS with 0.05% Tween-20 (PBST) and then incubated for 1 h in blocking solution (PBST with 5% milk). One hundred μL of skin explant (1:4) was further serially diluted out to 1:32 in 1× PBS on the horizontal axis of an antigen-coated ELISA plate and incubated at room temperature for 1 h or at 4° C. overnight. Plates were rinsed as described above and 100 μL of recombinant mouse monoclonal 9E1 antibody (Miller, Bly et al. 1987, Lange, Churchman et al. 2023) was added at 1:1000 dilution in blocking solution. After 1 h of incubation at room temperature, plates were washed with PBST and 100 μL of sheep anti-mouse IgG-HRP conjugated (Cytiva Life Sciences, Marlborough, MA) was diluted 1:5000 in blocking solution and incubated for 30 min at room temperature. Plates were rinsed three times with PBST, and 50 μL of 1-Step Ultra TMB-ELISA substrate solution (ThermoFisher, Waltham, MA) was added. The peroxidase reaction was stopped after 20 min with 50 μL of ELISA Stop Solution (ThermoFisher, Waltham, MA) and read spectrophotometrically at 490 nm with a Cytation 1 reader (Agilent, Santa Clara, CA).

Nine weeks post vaccination, the adjuvant control and vaccinated groups were challenged with wild type F. covae LV-359-01 with a calculated dose of 1.54×10⁷ CFU/mL. For the challenge four replicates of 25 catfish (˜500 g biomass/aquaria) were stocked into 40-L aquaria containing 10 L of water. Fish were not fed on the day before the laboratory challenge. An additional tank containing 5 fish from each treatment group were mock challenged. Fish were observed twice daily at which time any moribund fish were promptly removed.

Innate and immune gene-specific primers were utilized to independently evaluate the channel catfish skin immune response post vaccination via reverse transcription quantitative PCR (RT-qPCR). Each total RNA sample was assessed by using spectrophotometry (Bio-Tek Cytation 1, Agilent Technologies, Palo Alto, CA) and Agilent 2100 Bioanalyzer with RNA integrity numbers (RINs)>8 before reverse transcription. Then, cDNA synthesis was performed using the LunaScript® RT SuperMix Kit (New England Biolabs, Ipswich, MA, United States). Reactions contained 4.0 μL of LunaScript RT SuperMix (5×) and template RNA (200 ng), and the volume was adjusted using nuclease-free water to 20 μL. As a control, to rule out the presence of DNA in the extracted sample, no-RT reactions were prepared for each of the samples along with no template controls (negative control). Reaction conditions for cDNA synthesis included primer annealing at 25° C. for 2 min, cDNA synthesis at 55° C. for 10 min, and heat inactivation at 95° C. for 1 min. cDNA was kept at −20° C. after transcription until RT-qPCR.

Using a LightCycler® 480 System (Roche Diagnostics, Indianapolis, IN). The Luna Universal qPCR Master Mix was used for RT-qPCR in 10 μL reactions. Each reaction included 5 μL of Luna Universal qPCR Master Mix (2×), 0.5 μL of forward primer (1 μM), 0.5 μL of reverse primer (1 μM), 2 μL of nuclease free water, and 2 μL of cDNA diluted 1:10 with nuclease-free water. Reactions were carried out in triplicate under the following conditions: 95° C. for 15 s, followed by 45 cycles at 95° C. for 15 s, 60° C. for 30 s, followed by melting curve analysis. Reaction and cycling conditions were prepared following the manufacturer's instructions (NEB). The samples were run in parallel with two reference genes, 18S and EF1α for normalization. Relative gene expression was calculated using the 2-ΔΔCT method, normalizing with the geometric average of the reference gene relative to the controls (Livak & Schmittgen, Methods, (2001), 25:402-8; Pfaffl, M., Nucl. Acids. Res., (2011), 29:2003-7).

Survival data was analyzed using Kaplan-Meier log rank survival analysis and differences between vaccine groups and the control were determined using an unpaired, two-tailed t-test. P<0.05 were considered statistically significant. RT-qPCR data were evaluated by two-way analysis of variance (ANOVA) to determine differences in means among the control and vaccine groups and then further analyzed post hoc using Tukey's multiple comparison test. Differences of means among the groups were considered statistically significant when P<0.01. All data were analyzed and statistical tests were performed using GraphPad Prism version 10.1.2 (Prism, San Jose, California).

### Results

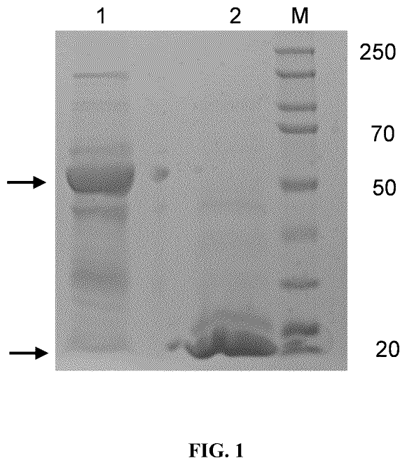

Based on our previous studies with F. covae recombinant proteins (Lange, Beck et al. 2016); the catalase and DPS proteins were expressed and purified using Ni-NTA agarose resin. The major eluted catalase and DPS proteins have migrated at their expected sizes, 56 and 22 kDa, respectively (FIG. 1). Both catalase and DPS are known to form oligomers, so the larger minor migrating bands for each may represent dimeric and trimeric forms of the recombinant proteins.

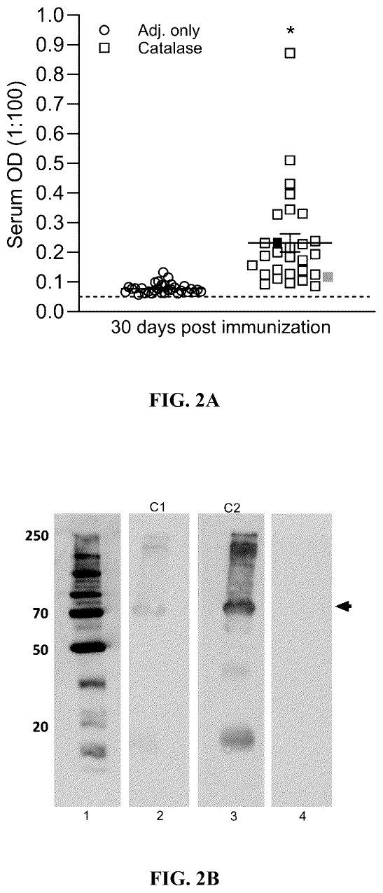

We initially sought to assess the immunogenicity of these F. covae proteins through intraperitoneal injection of different populations of fingerling catfish, including those administered catalase, DPS or adjuvant alone. Thirty days post immunization we sampled the catfish and collected blood. We screened the serum of non-immunized and immunized catfish which had been collected using an indirect ELISA. Absorbance values (0.086-0.87) demonstrate that individual catfish generated varying amounts of IgM antibodies to F. covae catalase as opposed to the adjuvant control with no detectable antibody response (FIG. 2A). An immunoblot of two individual catfish confirmed that the of IgM antibodies were to F. covae catalase (FIG. 2B).

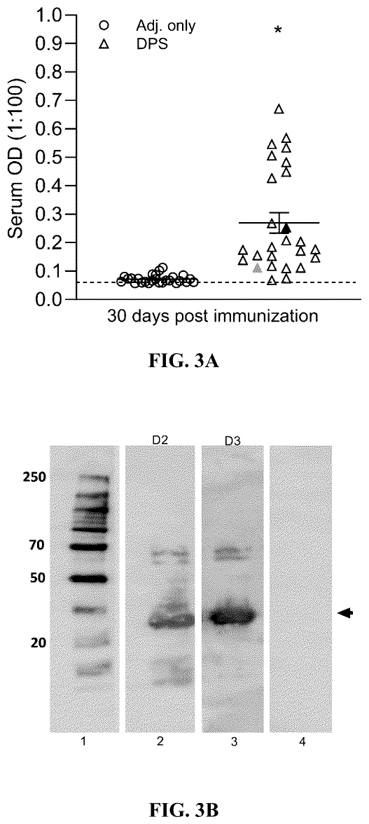

Absorbance values (0.068-0.67) demonstrated that individual fish generated varying amounts of IgM antibodies to F. covae DPS as opposed to the adjuvant control with no detectable antibody response (FIG. 3A). An immunoblot of two individual fish confirmed that the of IgM antibodies were to F. covae DPS (FIG. 3B).

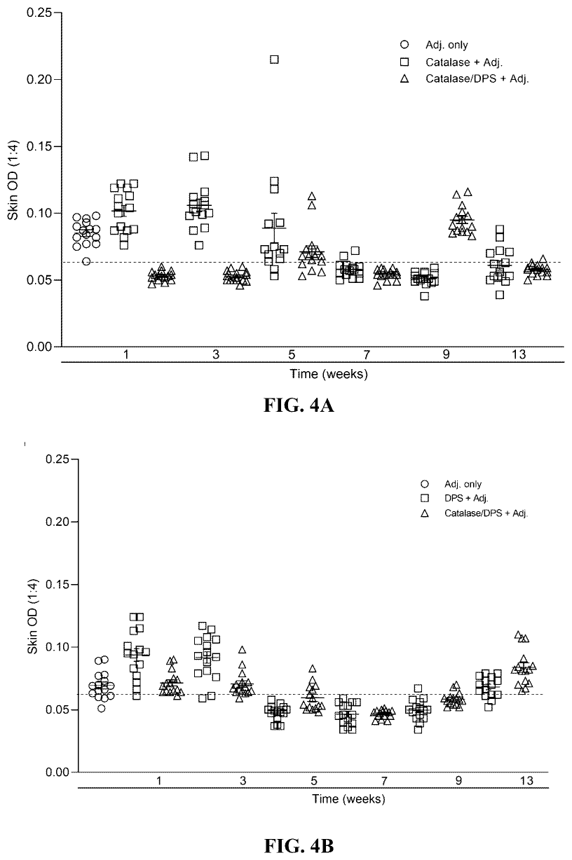

To determine the level at which mucosal IgM antibodies were generated to F. covae proteins, skin explant tissue culture medium was used to perform a catalase or DPS specific indirect ELISA. At 1-week post vaccination the adjuvant-only control had a mean absorbance value of 0.085, the catalase vaccinated group mean absorbance was 0.101 and the catalase/DPS group was 0.105 (FIG. 4A). The vaccinated groups demonstrated varying levels of Ab (0.088-0.094) over time that were higher than the adjuvant only control, however known were greater than two times the average background signal (0.06) across all plates assayed.

At 1-week post vaccination the adjuvant-only control had a mean absorbance value of 0.069, the DPS vaccinated group mean absorbance was 0.093 and the catalase/DPS group was 0.071 (FIG. 4B). The immunized groups demonstrated varying levels of Ab (0.071-0.083) over time that were higher than the adjuvant only control, however known were greater than two times the average background signal (0.06) across all plates assayed.

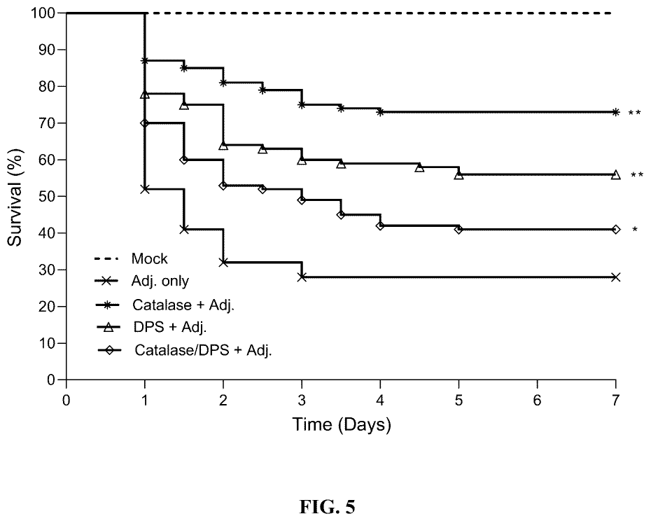

Nine weeks post vaccination we conducted a laboratory challenge on the non-immunized adjuvant control, catalase, DPS and catalase/DPS vaccinated groups with wild type F. covae LV-359-01. Kaplan-Meier survival analysis showed that 7 days post challenge there was a higher survival rate (P<0.05) among the three vaccinated groups when compared to the adjuvant control (FIG. 5). The catalase and DPS vaccinated groups were very significantly different with 73% and 56% survival and the combination of catalase/DPS was significantly different with 41% survival as compared to the 28% survival of the adjuvant control. These results indicate that significant protection was achieved with catalase and DPS, either individually or in combination.

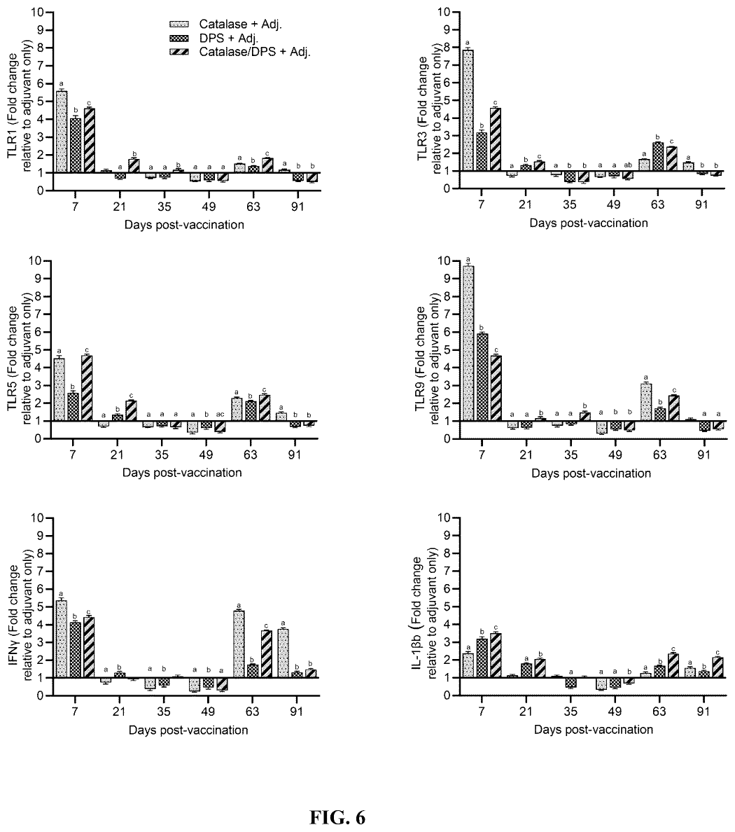

We performed gene expression analyses on skin samples from individual fish from the vaccine groups and the adjuvant only control, which served as the basal level of expression at each time point. After 7 days, we observed a very significant upregulation of TLR genes (TLR1, 3, 5 and 9) between 3-10-fold among the vaccine groups when compared to adjuvant control (FIG. 6). At the 21-49 day intervals the up-or downregulation of TRL genes was minimal, however at day 63 a very significant upregulation of TLR3, 5 and 9 genes was again observed in the vaccine groups. The catalase only group overall demonstrated much higher upregulation of TLR genes than the DPS or catalase/DPS groups.

We also evaluated the expression of proinflammatory cytokines, where we observed a very significant upregulation of IL-1βb and IFNγ 7-days post immunization in the vaccine groups when compared to the control. There was additional upregulation at later intervals (days 63-91) mostly with catalase group, but also to some degree in the catalase/DPS group. These results could suggest a priming of the innate immune response 7 days post immunization in the vaccine groups (specifically catalase) when compared to the control.

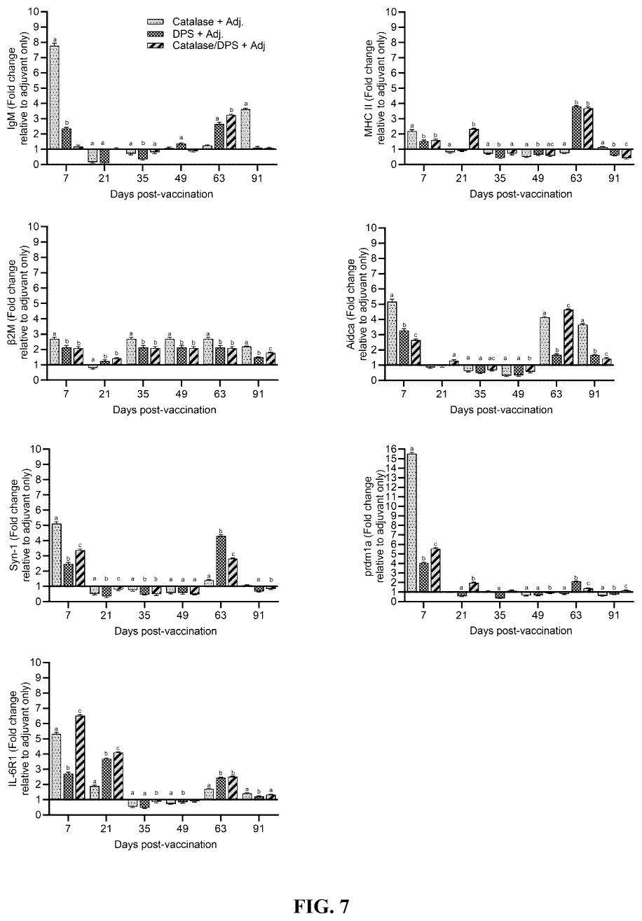

We next evaluated the expression of adaptive immune genes including those known to be expressed by B and T lymphocytes. We first assessed B-cell mediated genes, those we feel would most likely be involved in B cell processes occurring in the skin (FIG. 7). At seven days, we observed a very significant upregulation of the IgM gene, ˜2-8 fold in the catalase and DPS groups. There was no significant upregulation again until day 63 in the DPS and catalase/DPS groups and at day 91 where a significant upregulation was observed in the catalase group. At seven days, we observed a minimal significant upregulation of the MHC II gene, ˜2 fold among the vaccine groups. There was another significant upregulation on day 63 in the DPS only and catalase/DPS vaccine groups. The expression of β2M was consistent in its significant upregulation in the vaccine groups throughout the time course (˜2-fold). The Aidca gene was significantly upregulated (˜2.5-5-fold) at day 7 post vaccination in the vaccine groups. There was another significant upregulation on days 63 and 91 in each of the three vaccine groups. The Syn-1 gene was also significantly upregulated (˜2.5-5-fold) at day 7 post vaccination and again at day 63 in the vaccine groups. The prdm1A gene was significantly upregulated (˜4-16-fold) at day 7 post vaccination in the vaccine groups. and a minimal upregulation was observed at the later time points. Lastly, the IL-6R1 gene was significantly upregulated among each of the vaccine groups at 7 and 21 days (˜2-5-fold), and again at day 63.

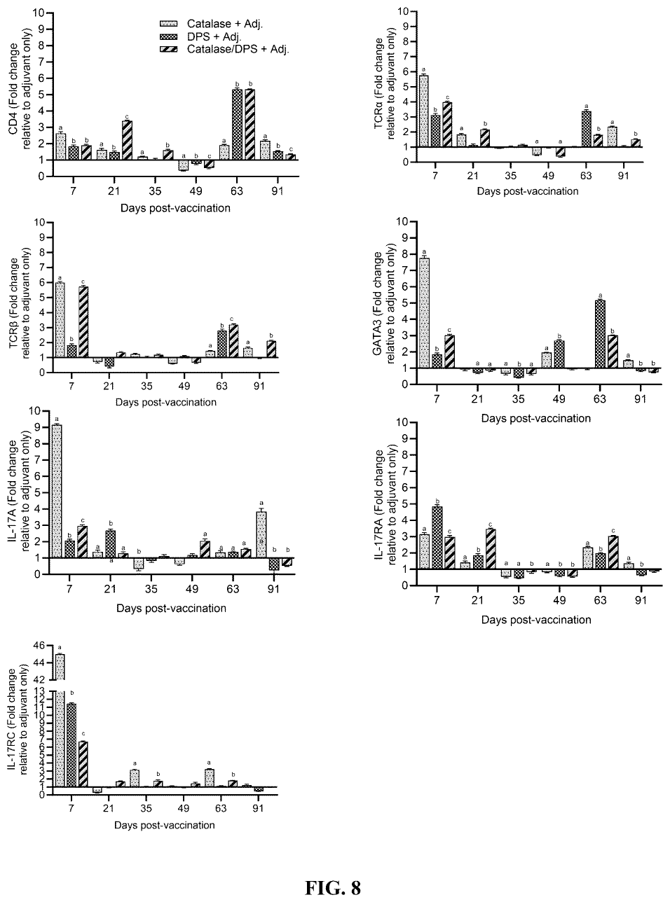

We next assessed T-cell mediated genes, those we feel would most likely be involved in T cell processes occurring in the skin (FIG. 8). The CD4 receptor gene was significantly upregulated among each of the vaccine groups at 7 and 21 days (˜2-fold), and again at day 63 (˜2-5-fold). The TCRα gene was significantly upregulated among each of the vaccine groups at 7 days (˜3-6-fold), and again at day 21, 63, 91 in different vaccine groups. The TCRβ gene shared a similar pattern to that of TCRα where it was significantly upregulated at day 7 in DPS group and very significantly in the catalase and catalase/DPS groups. At the later time points (day 63), TCRβ was very significantly upregulated in the DPS and catalase/DPS groups and significantly upregulated in the catalase and catalase/DPS groups on day 91. The GATA3 gene was significantly upregulated in each vaccine group at 7 days (˜2-8-fold), and again at day 63 (˜2-5-fold) and again variable at days 49, 63 and 91. The IL-17 genes (IL-17A, IL-17RA and Il-17RC) were all significantly upregulated in the vaccine groups at 7 days post vaccination ad was variable at days 49, 63 and 91. These results suggest that the activation of the adaptive immune system in the vaccine groups relative to the control has likely occurred.

### Example 2

### Vaccination Via Oral Delivery

All experimental fish protocols used in this study were approved by the USDA-ARS

Aquatic Animal Health Research Unit Institutional Animal Care and Use Committee.

The recombinant F. covae catalase and DPS proteins were expressed as described herein. Delta select channel catfish fingerlings were reared at the Aquatic Animal Health Research Unit, Auburn, AL, USA. 100 channel catfish (6 g) were stocked into nine 300 L tanks that received a mix of filtered well/municipal water and aeration from submerged air stones. The catfish were acclimated for 1-week and fed once daily 1% body weight. The F. covae recombinant protein vaccine(s) (SEQ ID NO: 2; SEQ ID NO: 6) were mixed with an oral adjuvant (Montanide GR01, SEPPIC, Fairfield, NJ) and then top-coated onto the feed. Commercially available fish feed was coated at 10% (w/w) with different concentrations of F. covae recombinant proteins (1, 10 and 20 μg/g feed). Separate lots of top-coated feed were generated for the oral adjuvant only and vaccine groups (catalase and DPS). The groups were orally vaccinated or received a placebo by feeding at a rate of 3% of body weight for 14 days. Fish were kept on flow through for 1-week post immunization and then all the tanks were switched over and maintained using a recirculating aquaculture system supplied with mixed (well and dechlorinated municipal water) for the remainder of the study.

Six weeks post vaccination, the control and vaccinated groups were challenged with wild type F. covae ALG-00-530 with a calculated dose of 4.40×10⁶ CFU/mL. For the challenge, four replicates of 20 catfish were stocked into 40-L aquaria containing 10 L of water. Fish were not fed on the day before the laboratory challenge. An additional tank containing 3 fish from the adjuvant control group and each vaccine group were mock challenged (n=21). Fish were observed twice daily at which time any moribund fish were promptly removed.

Survival data was analyzed using Kaplan-Meier log rank survival analysis. Probabilities of 0.05 or less were considered statistically significant. All statistical tests were performed using GraphPad Prism version 10.3.1 (Prism, San Jose, California).

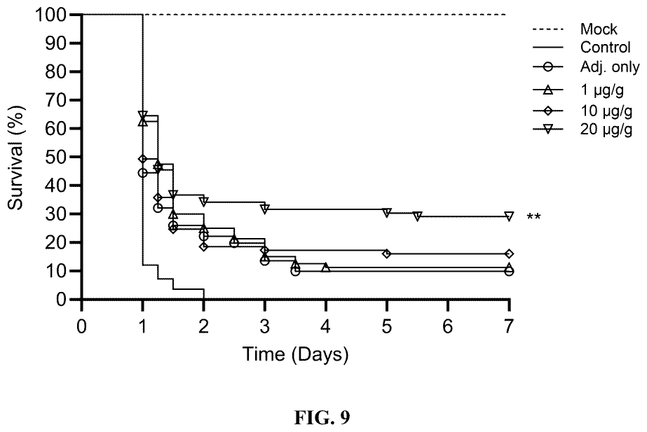

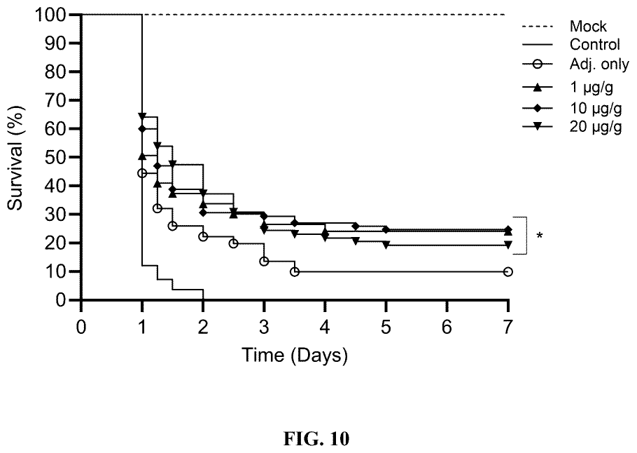

Efficacy of F. covae recombinant protein vaccines against columnaris disease was also assessed. Six weeks post vaccination we conducted a laboratory challenge on the non-immunized controls, and the catalase (SEQ ID NO:2) and DPS (SEQ ID NO:6) vaccinated groups with wild type F. covae ALG-00-530. Kaplan-Meier survival analysis showed that 7-days post-challenge the control group had 0% survival, and the adjuvant had 10% survival. The catalase-vaccinated groups (1, 10 and 20 μg/g) had 12, 16 and 30% survival respectively. The highest oral dose (20 μg/g) had a very significantly higher survival rate (P <0.05) than the adjuvant only group (FIG. 9). The DPS-vaccinated groups (1, 10 and 20 μg/g catalase) had 20, 23 and 20% survival respectively. All three oral doses (DPS) had a significantly higher survival rate (P<0.05) than the adjuvant only group (FIG. 10). These initial trials demonstrate the efficacy of F. covae recombinant protein vaccines (catalase and DPS) when delivered orally. Additional work will continue to optimize the oral dose and timing of immunization which could include booster immunization to maximize the effectiveness of these recombinant protein vaccines.

While the invention has been described with reference to details of the illustrated embodiments, these details are not intended to limit the scope of the invention as defined in the appended claims. The embodiment of the invention in which exclusive property or privilege is claimed is defined as follows:

What is claimed is:

1. A composition comprising an isolated protein having an amino acid sequence at least 75% identical to SEQ ID NO: 2, SEQ ID NO: 6, or a combination thereof, and an adjuvant.

2. The composition of claim 1, wherein the protein is at least 75% identical to SEQ ID NO: 2.

3. The composition of claim 1, wherein the protein is at least 75% identical to SEQ ID NO: 6.

4. A composition comprising an isolated protein having an amino acid sequence at least 75% identical to SEQ ID NO: 2 and an isolated protein having an amino acid sequence at least 75% identical to SEQ ID NO: 6.

5. The composition of claim 4, further comprising an adjuvant.

6. A vaccine composition comprising an effective amount of a protein having an amino acid sequence at least 75% identical to SEQ ID NO: 2, SEQ ID NO: 6, or a combination thereof, and an adjuvant.

7. The vaccine of claim 6, wherein the protein is at least 75% identical to SEQ ID NO:2.

8. The vaccine of claim 6, wherein the protein is at least 75% identical to SEQ ID NO:6.

9. The vaccine of claim 6, wherein the vaccine comprises an isolated protein having an amino acid sequence at least 75% identical to SEQ ID NO: 2 and an isolated protein having an amino acid sequence at least 75% identical to SEQ ID NO: 6.

10. An expression vector comprising a promoter and a heterologous polynucleotide, wherein the heterologous polynucleotide encodes a protein having an amino acid sequence at least 75% identical to SEQ ID NO: 2 or SEQ ID NO: 6, and wherein the promoter is operatively linked to the heterologous polynucleotide.

11. A method of eliciting an immune response against Flavobacterium covae in a subject, comprising administering to the subject a composition comprising a protein at least 75% identical to SEQ ID NO: 2 or SEQ ID NO: 6, thereby eliciting an immune response to F. covae.

12. The method of claim 11, wherein the protein comprises SEQ ID NO: 2 or SEQ ID NO: 6.

13. The method of claim 11, wherein the administered composition comprises two proteins, wherein the first protein is at least 75% identical to SEQ ID NO: 2 and the second protein is at least 75% identical to SEQ ID NO: 6.

14. The method of claim 11, wherein the composition further comprises a pharmaceutically acceptable carrier, an adjuvant, or both a pharmaceutically acceptable carrier and an adjuvant.

15. The method of claim 11, wherein the subject is a fish.

16. The method of claim 15, wherein the fish is a catfish.

17. The method of claim 11, wherein the administering is oral administering.
